(19) United States
(12) Patent Application Publication
(10) Pub. No.: US 2026/0026855 A1
(43) Pub. Date: Jan. 29, 2026

(54) FRONTAL SINUS FRACTURE CLOSED REDUCTION INSTRUMENTS

(71) Applicant: THE UAB RESEARCH FOUNDATION, BIRMINGHAM, AL (US)

(72) Inventors: Bradford Alan Woodworth, Birmingham, AL (US); Harishanker Jeyarajan, Mountain Brook, AL (US)

(21) Appl. No.: 19/283,414

(22) Filed: Jul. 29, 2025

Related U.S. Application Data
(60) Provisional application No. 63/676,502, filed on Jul. 29, 2024.

(51) Int. Cl. A61B 17/88; A61B 17/00; A61B 17/24; A61B 90/00

(52) U.S. Cl. CPC A61B 17/8866; A61B 17/24; A61B 90/08; A61B 2017/00792; A61B 2017/00831; A61B 2017/00982

(57) ABSTRACT

Various surgical instruments for closed reduction of the frontal sinus anterior table, a kit of surgical instruments, and method of use are described. The surgical instrument includes a handle defining a proximal portion of the surgical reduction instrument and an elongated shaft extending from the handle to a tip end at a distal end of the surgical reduction instrument. The elongated shaft includes a base portion and a curved portion, the base portion extending along an axis and having a substantially constant cross section, the curved portion having a taper portion and a tip portion, the tip portion being substantially flat having a thickness and a width ending at the tip end, the taper portion providing transition between the base portion and the tip portion, and the tip end suitable for reducing a fracture of an anterior table of an individual.

### CROSS-REFERENCE TO RELATED APPLICATIONS

This application claims benefit of U.S. Provisional Application No. 63/676,502, filed on Jul. 29, 2024, which is hereby incorporated by reference in its entirety.

### BACKGROUND

Facial trauma may result in maxillofacial fractures that can involve the frontal bar and/or sinuses. The majority of frontal sinus fractures involve the anterior table, whether in isolation or in conjunction with posterior table involvement.

### BRIEF DESCRIPTION OF THE DRAWINGS

FIG. 1 shows an example frontal sinus fracture closed reduction instrument according to various aspects of the embodiments.

FIG. 2 shows front and side views of an example tip portion of the fracture reduction instrument of FIG. 1 according to various aspects of the embodiments.

FIG. 3 shows a side view of an alternate example tip portion of a fracture reduction instrument of FIG. 1 according to various aspects of the embodiments.

FIG. 4 shows example side views of the frontal sinus fracture reduction instrument of FIG. 1 with various curved portions according to various aspects of the embodiments.

FIG. 5 shows example side views of frontal sinus fracture reduction instrument of FIG. 1 according to various aspects of the embodiments.

FIGS. 6A-F shows examples of use of fracture reduction instrument of FIG. 1 to reduce the fracture transnasally of an example patient according to various aspects of the embodiments.

### DETAILED DESCRIPTION

Approximately 5-15% of maxillofacial fractures involve the frontal bar and/or sinuses. The majority of frontal sinus fractures involve the anterior table, whether in isolation or in conjunction with posterior table involvement. Isolated posterior table fractures are uncommon; instead, these are often coupled with an anterior table fracture. Treatment of frontal sinus fractures carries a unique complexity in comparison to other types of facial fractures which is reflected in the numerous classification and management algorithms in the literature. Traditionally, open approaches were the mainstay of management for frontal sinus anterior table fractures and would include bi-coronal or direct glabellar incisions to access the frontal sinus for open reduction and internal fixation (ORIF). More recently, the transnasal endoscopic approach has been incorporated as a suitable option in the surgical management algorithm.

The management of frontal sinus fractures has remained an area of controversy for the better part of the 20th and 21st century with many authors recommending various algorithms that include conservative, open, and endoscopic techniques. Traditional management of both anterior and posterior table frontal fractures involves aggressive open techniques including cranialization, sinus obliteration, and/or osteoplastic flap. With the advent of endoscopic techniques, there is now a paradigm shift from open techniques in favor of conservative and minimally invasive methods. Advantages of the transnasal endoscopic technique for frontal sinus fractures is similar to those previously described in sinonasal tumor resection and treatment of other frontal sinus pathologies. Many studies have shown reduced external scarring, length of stay, and operative time with overall similar or improved outcomes with endoscopic approaches.

Restoration of the frontal bone contour is the largest component in cosmetic outcomes particularly in cases with >2-6 mm displacement of the anterior table. The paramedian location of the frontal sinuses allows a cosmetic defect to become a prominent focus of attention. Importantly, excellent reduction with superb FACE-Q cosmetic satisfaction outcomes using the transnasal endoscopic technique was recently shown. Endoscopic repair of frontal sinus fractures eliminates external surgical scarring, removes the need for hardware implantation, results in excellent contour with minimal residual displacement, and patients reported outstanding cosmetic quality of life in our study. A transnasal endoscopic approach will likely lead to a reduction in postoperative complications and need for revision surgeries following frontal sinus fracture repair.

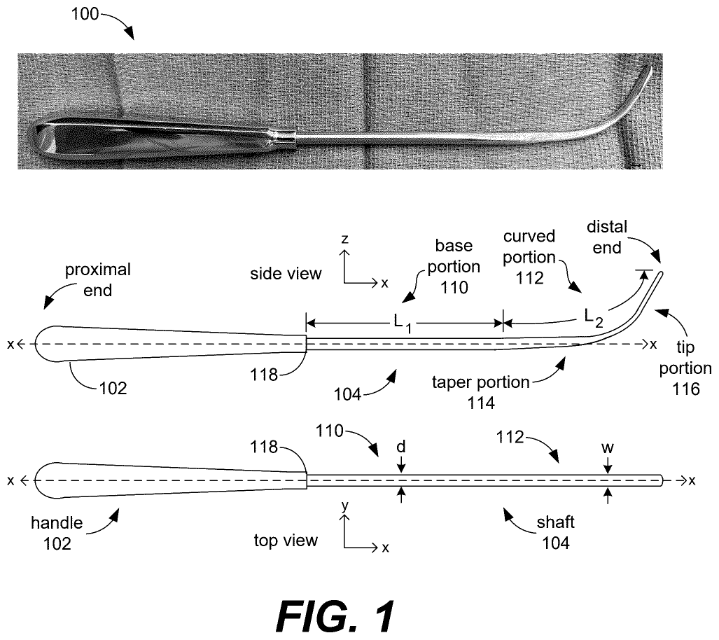

In the context described above, various examples of frontal sinus fracture closed reduction instruments (also referred to as fracture reduction instruments, anterior table fracture reduction instruments, reducers, and surgical instruments herein) and methods of use for closed reduction of the frontal sinus anterior table are described herein. As shown in the example in FIG. 1, frontal sinus fracture closed reduction instruments 100 have been developed to simplify elevation of fractures and improve the capability to reach superior and lateral fractures. These instruments are stronger than frontal curettes and curved suction instruments and provide a broader surface area to reduce the fracture without introducing additional fractures. Several different shapes and angles to the reducers have been developed to extend capabilities.

As shown in FIG. 1, the frontal sinus fracture closed reduction instrument 100 includes a handle 102 defining a proximal portion of the fracture reduction instrument 100 and an elongated shaft 104 extending from the handle 102 to a tip end 106 at a distal end of the fracture reduction instrument 100. The elongated shaft 104 comprises a base portion 110 and a curved portion 112. The base portion 110 extending along an axis (x-x) and having a substantially constant cross section (not shown). For example, the cross-section of the base portion can be substantially round; however, other shapes and cross sections can be relied upon. The curved portion 112 comprising a taper portion 114 and a tip portion 116. The tip portion 116 being substantially flat having a thickness (t) and a width (w) ending at the blunt tip. The taper portion 114 providing transition in cross section between the base portion 110 and the tip portion 116. The tip end 106 being suitable for reducing a fracture of an anterior table of an individual. In various examples of the present disclosure, the tip end 106 can take a number of different shapes and configurations such as a smoothed tip, a toothed tip, a serrated tip, a fenestrated tip, a curved tip, an angled tip, a blunt tip, a rounded tip, a hooked tip, a concaved tip, a bulbous tip, and any other suitable configuration for reducing a fracture of an anterior table. The shaft 104 can be formed of a biocompatible material. For example, the shaft 104 can be formed of a stainless steel, or other suitable metals or materials that can be sterilized for surgical use. The elongated shaft 104 can be formed in one piece. In some examples, the elongated shaft 104 and the handle 102 are formed in one piece.

As shown in FIG. 1, the handle 102 of the fracture reduction instrument 100 is suitable to grasp with a hand to direct and apply pressure of the tip end 106 at the distal end of the fracture reduction instrument 100. While the handle 102 is shown as symmetrical about the axis (x-x) of the fracture reduction instrument 100, other shapes and configurations can be relied on. For example, the handle 102 can have an ergonomic design to provide a better grip and/or leverage for the surgeon.

The elongated shaft 104 of the fracture reduction instrument 100 can have a shaft length (L_{T}) from the handle 102 to the tip end 106 of about 5 to about 12 inches. The shaft length comprises a base portion length (L₁) and a curved portion length (L₂). The base portion can have a length of about 4 inches to about 6 inches. The curved portion can have a length of about 1.25 inches to about 5.5 inches. In some examples, the elongated shaft 104 can have a portion embedded partially or fully in the handle 102; however, the shaft length (L_{T}) is measured from a collar 118 of the handle 102 from which the elongated shaft 104 extends.

The base portion 110 of the elongated shaft 104 extends from the handle 102 along the axis (x-x). In some examples, the base portion 110 can extend in the same direction as the axis (x-x) but be offset from the axis (x-x) and/or handle 102. In some examples, the base portion 110 can extend along the axis (x-x) with the handle 102 having an incident angle with respect to the axis (x-x) and in relation to the curvature of the tip portion 116.

The curved portion 112 of the elongated shaft 104 extends from the base portion 110. The curved portion 112 comprising a tip portion 116 and the tip end 106. The curved portion length (L₂) can be measured from the position along the elongated shaft 104 where the shaft begins to curve, bend, or angle away from the axis. In some examples, the curved portion 112 comprises a taper portion 114 to provide a transition from the shape and/or cross-section of the base portion 110 to the tip portion 116. In some examples, the shape and/or cross-section of the base portion 110 can begin a transition at a position along the axis proximal to the beginning of the curved portion 112. As shown in FIG. 1, the diameter (d) or width of the base portion 110 can be substantially the same as the width (w) of the tip portion 116. In some examples, the width (w) of the tip portion 116 can be greater than the diameter (d) or width of the base portion 110.

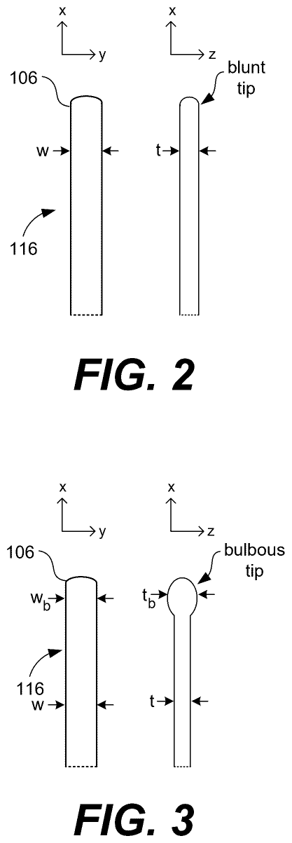

Shown in FIG. 2 are front and side views of an example of the tip portion 116 of the fracture reduction instrument 100. The tip end 106 can be slightly rounded and suitable to apply pressure to a displaced fragment or section for reduction without introducing additional fractures of the anterior table. As shown, the width (w) of the tip portion 116 can be substantially constant and the thickness (t) providing a substantially flat surface. For example, the thickness (t) can be about 1.5 mm. In an example, the tip portion 116 can have two substantially parallel flat surfaces. For example, the cross-section of the tip portion 116 can be substantially rectangular with rounded edges or have a flattened oval shape, among other shapes. In another example, one surface of the tip portion 116 can be substantially flat and the cross-section shaped to provide suitable support so that the shaft 104 does not bend when a force is applied to reduce the fracture.

Shown in FIG. 3 are example front and side views of another example of the tip portion 116 of the fracture reduction instrument 100. In this example, the tip end 106 may include a bulbous tip. Instead of flattening out and terminating such that the tip end is substantially flat or slightly rounded as depicted in FIG. 2, the tip portion flattens out and then bulges at the tip end 106 to create a bulbous working surface. As shown, the width (w) of the tip portion 116 can be substantially constant and the thickness (t) providing a substantially flat surface. In this example, as shown, the bulbous tip has a thickness (t_{b}) greater than the thickness of the tip portion (t). However, the width (w_{b}) of the tip portion 116 at the bulbous tip can be substantially the same as the width (w) of the tip portion 116. In some examples, width (w_{b}) of the tip portion 116 at the bulbous tip can be greater than the width (w) of the tip portion 116.

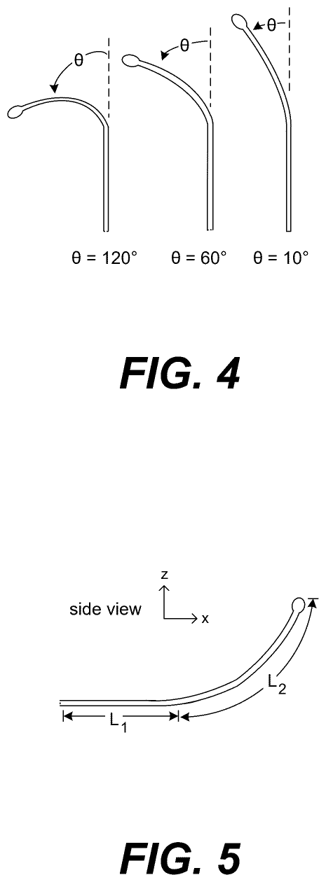

As shown in FIG. 4, the fracture reduction instrument 100 can be configured such that the tip end 106 is positioned at an angle (θ) relative to the base portion 110 of the elongated shaft 104. The angle of curvature at the distal end of the surgical instrument can be sharper or broader depending on the need. For example, individual ones of the surgical instrument can be configured with different curvatures or angles to permit various approaches to the frontal recess during a procedure to reduce the anterior table fracture. The blunt tip of the curved portion can be formed with an angle of about 10 degrees to about 120 degrees from the axis. It should be noted that the curvature of the curved portion 112 is not intended to bend or bend during use. A series of fracture reduction instruments 100 having different upper angle profiles can be provided in a kit to allow permit an outstanding approach to the frontal recess based on the specific need.

FIG. 5 provides an additional example of a side view of the elongated shaft 104 showing a bulbous tip. The length (L₁) of base portion 110 of the shaft can be 4 to 6 inches. The length (L₂) of curved portion 112 of the shaft can be from about 1.25 inches to about 5.5 inches. A series of fracture reduction instruments 100 having different lengths of base and/or curved portions can be provided in a kit to allow permit an outstanding approach to the frontal recess based on the specific need.

In an example, various surgical fracture reduction instruments 100 having a range of dimensions can be provided as a kit so that the surgeon can select from several different sizes, shapes, and angles of a fracture reduction instrument 100 from a plurality of fracture reduction instruments 100. For example, several different angles, lengths, and widths to enable a range of positioning within the sinus can be provided. For example, the kit can include a plurality of surgical reduction instruments, with individual ones of the plurality of surgical reduction instruments differing in at least one dimension from the other surgical reduction instruments.

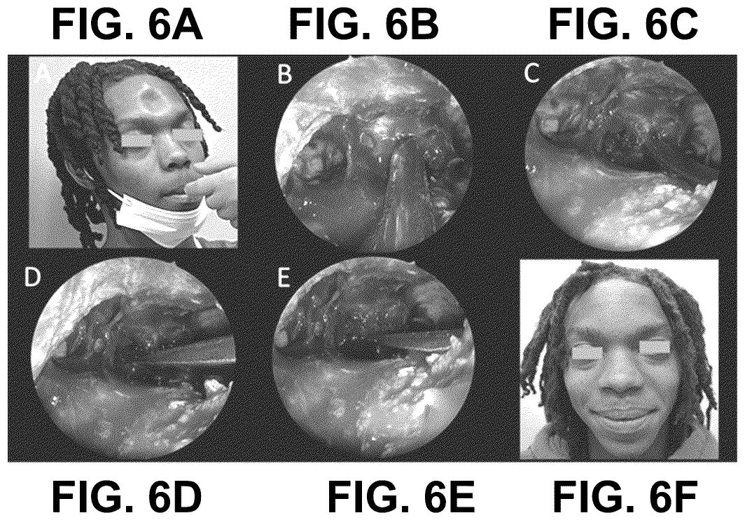

In FIGS. 6A-6F, an example of the method of use of the surgical instrument for closed reduction of the frontal sinus anterior table is shown. In FIG. 6A, a patient with a severely displaced anterior table fracture with a large divot in the forehead is shown before surgery. In this example, the method includes, visualizing with a 70-degree endoscope, the reduction instrument is introduced through the Draf 3 opening at the base of the frontal sinus and is placed on the inferior segment, as shown in FIG. 6B. Next, as shown in FIG. 6C, the segment is reduced. Then, as shown in FIG. 6D, the superior segment is addressed. The example view of FIG. 6E shows the fracture segments are now in reduction. In FIG. 6F, a postoperative clinic view of the patient with a normal contour to the forehead is shown.

The basic technique for fracture reduction is as follows. In a portion of cases, a Draf 2B or Draf 3 frontal sinusotomy is generally used for the approach to provide a wide aperture to the base of the frontal sinus for insertion of the instruments. Using a 70-degree endoscope, insert the instrument through the Draf 2B or Draf 3 opening, reduce the inferior or medial sections of the fracture first, and then reduce the superior or lateral fragments. Then, visualize the reduction endoscopically and palpate the forehead to confirm adequate placement of the fracture segments. Although Draf 2B and Draf 3 openings are provided as an example of transnasal approaches, the frontal sinus fracture closed reduction instruments can also be relied on for other surgical approaches such as, a Draf 2A or a different surgical incision such as a Lynch incision or frontal trephination. Additionally, in some examples it is possible to utilize the frontal sinus fracture closed reduction instruments without an endoscope for visualization.

In an example, the surgical method for reducing a fracture of an anterior table of an individual can include forming an opening at a base of a frontal sinus of the individual. Forming the opening at the base of the frontal sinus can involve any frontal sinus opening through the nose through a frontal sinusotomy (usually a Draf 2A, 2B or a Draf 3 frontal sinusotomy), but can also be approached through an incision such as a Lynch incision or frontal trephination. In some examples, the method can also include visualizing the fracture. For example, an endoscope can be used for visualizing the fracture, as well as the position of the fracture reduction instrument 100. In some examples, the fracture reduction instruments 100 can be used to reduce a fracture without the use of an endoscope. The method also includes introducing a fracture reduction instrument 100 though the opening at the base of the frontal sinus. The method also includes identifying at least one segment of the anterior table fracture. For example, at least a section or a portion of one or more fragments of the anterior table fracture can be identified. The method also includes applying a pressure with a blunt tip of the fracture reduction instrument 100 to reduce the at least one segment. For example, reducing the at least one segment can include reducing one or more inferior or medial segments prior to reducing one or more superior or lateral segments.

The above-described examples of the present disclosure are merely possible examples of implementations set forth for a clear understanding of the principles of the disclosure. Many variations and modifications can be made without departing substantially from the spirit and principles of the disclosure. All such modifications and variations are intended to be included herein within the scope of this disclosure and protected by the following claims.

### REFERENCES

1. Kim I A, Boahene K D, Byrne P J. Trauma in Facial Plastic Surgery: Frontal Sinus Fractures. Facial Plast Surg Clin North Am 2017; 25:503-511.
2. Grayson J W, Jeyarajan H, Illing E A, Cho D Y, Riley K O, Woodworth B A. Changing the surgical dogma in frontal sinus trauma: transnasal endoscopic repair. International forum of allergy & rhinology 2017; 7:441-449.
3. Strong E B, Pahlavan N, Saito D. Frontal sinus fractures: a 28-year retrospective review. Otolaryngol Head Neck Surg 2006; 135:774-779.
4. Banks C, Grayson J, Cho D Y, Woodworth B A. Frontal sinus fractures and cerebrospinal fluid leaks: a change in surgical paradigm. Current opinion in otolaryngology & head and neck surgery 2020; 28:52-60.
5. Wang E W, Zanation A M, Gardner P A et al. ICAR: endoscopic skull-base surgery. International forum of allergy & rhinology 2019; 9: S145-S365.
6. Jones V, Virgin F, Riley K, Woodworth B A. Changing paradigms in frontal sinus cerebrospinal fluid leak repair. International forum of allergy & rhinology 2012; 2:227-232.
7. Woodworth B A, Harvey R J, Neal J G, Palmer J N, Schlosser R J. Endoscopic management of frontal sinus mucoeceles with anterior table erosion. Rhinology 2008; 46:231-237.
8. Woodworth B A, Schlosser R J, Palmer J N. Endoscopic repair of frontal sinus cerebrospinal fluid leaks. The Journal of laryngology and otology 2005; 119:709-713.
9. Virgin F, Baranano C F, Riley K, Woodworth B A. Frontal sinus skull base defect repair using the pedicled nasoseptal flap. Otolaryngology—head and neck surgery: official journal of American Academy of Otolaryngology-Head and Neck Surgery 2011; 145:338-340.
10. Virgin F W, Bleier B S, Woodworth B A. Evolving materials and techniques for endoscopic sinus surgery. Otolaryngologic clinics of North America 2010; 43:653-672, xi.
11. Smith N, Riley K O, Woodworth B A. Endoscopic Coblator-assisted management of encephaloceles. The Laryngoscope 2010; 120:2535-2539.
12. Arnold M A, Tatum S A, 3rd. Frontal Sinus Fractures: Evolving Clinical Considerations and Surgical Approaches. Craniomaxillofac Trauma Reconstr 2019; 12:85-94.
13. Choi K J, Chang B, Woodard C R, Powers D B, Marcus J R, Puscas L. Survey of Current Practice Patterns in the Management of Frontal Sinus Fractures. Craniomaxillofac Trauma Reconstr 2017; 10:106-116.
14. Dedhia R D, Morisada M V, Tollefson T T, Strong E B. Contemporary management of frontal sinus fractures. Curr Opin Otolaryngol Head Neck Surg 2019; 27:253-260.
15. Schultz J J, Chen J, Sabharwal S et al. Management of Frontal Bone Fractures. J Craniofac Surg 2019; 30:2026-2029.
16. Banks C G, Garcia J A P, Grayson J, Yeon Cho D, Woodworth B A. Osteoplastic Flap Without Obliteration: How I Do It. American journal of rhinology & allergy 2018; 32:346-349.
17. Chaaban M R, Conger B, Riley K O, Woodworth B A. Transnasal endoscopic repair of posterior table fractures. Otolaryngology—head and neck surgery: official journal of American Academy of Otolaryngology-Head and Neck Surgery 2012; 147:1142-1147.
18. Conger B T, Jr., Illing E, Bush B, Woodworth B A. Management of Lateral Frontal Sinus Pathology in the Endoscopic Era. Otolaryngology—head and neck surgery: official journal of American Academy of Otolaryngology-Head and Neck Surgery 2014; 151:159-163.
19. Illing E A, Woodworth B A. Management of Frontal Sinus Cerebrospinal Fluid Leaks and Encephaloceles. Otolaryngologic clinics of North America 2016; 49:1035-1050.
20. Tilak A, Purvis J, Pena-Garcia A et al. Above and Beyond: Periorbital Suspension for Endoscopic Access to Difficult Frontal Sinus Pathology. The Laryngoscope 2021.
21. Lu V M, Ravindran K, Phan K et al. Surgical Outcomes of Endoscopic Versus Open Resection for Primary Sinonasal Malignancy: A Meta-analysis. Am J Rhinol Allergy 2019; 33:608-616.
22. Povolotskiy R, Farber N I, Bavier R D, Cerasiello S Y, Eloy J A, Hsueh W D. Endoscopic Versus Open Resection of Non-Squamous Cell Carcinoma Sinonasal Malignancies. Laryngoscope 2020; 130:1872-1876.
23. Santos P L D, Chihara L L, Alcalde L F A, Masalskas B F, Sant'Ana E, Faria P E P. Outcomes in Surgical Treatment of Mucocele in Frontal Sinus. J Craniofac Surg 2017; 28:1702-1708.
24. Yin G, Guo W, Chen X, Huang Z. Prognosis of endoscopic surgery and traditional open resection in mucosal melanoma of the nasal cavity and paranasal sinus. Melanoma Res 2019; 29:47-52.
25. Allensworth J J, Rowan N R, Storck K A, Woodworth B A, Schlosser R J. Endoscopic repair of spontaneous skull base defects decreases the incidence rate of intracranial complications. International forum of allergy & rhinology 2019; 9:1089-1096.
26. Shahangian A, Soler Z M, Baker A et al. Successful repair of intraoperative cerebrospinal fluid leaks improves outcomes in endoscopic skull base surgery. International forum of allergy & rhinology 2017; 7:80-86.
27. Harvey R J, Nalavenkata S, Sacks R et al. Survival outcomes for stage-matched endoscopic and open resection of olfactory neuroblastoma. Head & neck 2017; 39:2425-2432.
28. Nalavenkata S B, Sacks R, Adappa N D et al. Olfactory Neuroblastoma: Fate of the Neck—A Long-term Multicenter Retrospective Study. Otolaryngology—head and neck surgery: official journal of American Academy of Otolaryngology-Head and Neck Surgery 2016; 154:383-389.
29. Grayson J W, Khichi S S, Cho D Y, Riley K O, Woodworth B A. Management Strategies for Skull Base Inverted Papilloma. Otolaryngology—head and neck surgery: official journal of American Academy of Otolaryngology-Head and Neck Surgery 2016; 155:179-183.
30. Walsh E, Illing E, Riley K O et al. Inaccurate Assessments of Anterior Cranial Base Malignancy Following Nasoseptal Flap Reconstruction. Journal of neurological surgery Part B, Skull base 2015; 76:385-389.
31. Illing E, Chaaban M R, Riley K O, Woodworth B A. Porcine small intestine submucosal graft for endoscopic skull base reconstruction. International forum of allergy & rhinology 2013; 3:928-932.
32. Chaaban M R, Woodworth B A, Vattoth S, Tubbs R S, Owen Riley K. Surgical approaches to central skull base and postsurgical imaging. Seminars in ultrasound, C T, and M R 2013; 34:476-489.
33. Chaaban M R, Woodworth B A. Complications of skull base reconstruction. Advances in oto-rhino-laryngology 2013; 74:148-162.
34. Blount A, Riley K, Cure J, Woodworth B A. Cerebrospinal fluid volume replacement following large endoscopic anterior cranial base resection. International forum of allergy & rhinology 2012; 2:217-221.
35. Blount A, Riley K O, Woodworth B A. Juvenile nasopharyngeal angiofibroma. Otolaryngologic clinics of North America 2011; 44:989-1004, ix.
36. Kostrzewa J P, Sunde J, Riley K O, Woodworth B A. Radiofrequency coblation decreases blood loss during endoscopic sinonasal and skull base tumor removal. ORL; journal for oto-rhino-laryngology and its related specialties 2010; 72:38-43.
37. Strong E B. Frontal sinus fractures: current concepts. Craniomaxillofac Trauma Reconstr 2009; 2:161-175.

1. A surgical instrument, comprising:
a handle defining a proximal portion of the surgical instrument; and
an elongated shaft extending from the handle to a tip end at a distal end of the surgical instrument, the elongated shaft comprising a base portion and a curved portion, the base portion extending along an axis and having a substantially constant cross section, the curved portion having a taper portion and a tip portion, the tip portion being substantially flat having a thickness and a width ending at the tip end, the taper portion providing transition between the base portion and the tip portion, and the tip end suitable for reducing a fracture of an anterior table of an individual.

2. The surgical instrument of claim 1, wherein the elongated shaft has a shaft length from the handle to the tip end of about 5 to about 12 inches.

3. The surgical instrument of claim 1, wherein the base portion has a length of about 4 inches to about 6 inches.

4. The surgical instrument of claim 1, wherein the curved portion has a length of about 1.25 inches to about 5.5 inches.

5. The surgical instrument of claim 1, wherein the tip end of the curved portion is bent to an angle of about 10 degrees to about 120 degrees from the axis.

6. The surgical instrument of claim 1, wherein the tip end comprises one of at least a smoothed tip, a toothed tip, a serrated tip, a fenestrated tip, a curved tip, an angled tip, a blunt tip, a rounded tip, a hooked tip, a concaved tip, or a bulbous tip.

7. The surgical instrument of claim 1, wherein the shaft is formed of a biocompatible material.

8. The surgical instrument of claim 1, wherein the shaft is formed of a stainless steel.

9. A kit of surgical instruments, comprising:
a plurality of surgical instruments, individual ones of the plurality of surgical instruments differing in at least one dimension from the other surgical instruments, each of said surgical instrument comprising:
a handle defining a proximal portion of the surgical instrument; and
an elongated shaft extending from the handle to a tip end at a distal end of the surgical instrument, the elongated shaft comprising a base portion and a curved portion, the base portion extending along an axis and having a substantially constant cross section, the curved portion having a taper portion and a tip portion, the tip portion being substantially flat having a thickness and a width ending at the tip end, the taper portion providing transition between the base portion and the tip portion, and the tip end suitable for reducing a fracture of an anterior table of an individual;
wherein the at least one dimension is selected from one or more of: a base portion length, a curved portion length, a width of the tip portion, an angle of the tip end with respect to the axis, and a thickness of the tip end.

10. The kit of surgical instruments of claim 9, wherein individual ones of the plurality of surgical instruments have the base portion length of about 4 inches to about 6 inches.

11. The kit of surgical instruments of claim 9, wherein individual ones of the plurality of surgical instruments have the curved portion length of about 1.25 inches to about 5.5 inches.

12. The kit of surgical instruments of claim 9, wherein individual ones of the plurality of surgical instruments have the angle of the tip end curved with respect to the axis at about 10 degrees to about 120 degrees.

13. The kit of surgical instruments of claim 9, wherein at least one of the individual surgical instruments has a blunt tip end and at least one of the individual surgical instruments has a bulbous tip end.

14. A surgical method for reducing a fracture of an anterior table of an individual, comprising:
forming an opening to a frontal sinus of the individual;
introducing a surgical instrument though the opening at a base of the frontal sinus;
identifying at least one segment of the anterior table fracture;
applying a pressure with a tip end of the surgical instrument to reduce the at least one segment; and
wherein the surgical instrument comprises: a handle defining a proximal portion of the surgical instrument and an elongated shaft extending from the handle to a tip end at a distal end of the surgical instrument, the elongated shaft comprising a base portion and a curved portion, the base portion extending along an axis and having a substantially constant cross section, the curved portion having a taper portion and a tip portion, the tip portion being substantially flat having a thickness and a width ending at the tip end, the taper portion providing transition between the base portion and the tip portion, and the tip end suitable for reducing a fracture of an anterior table of an individual.

15. The surgical method of claim 14, wherein identifying the at least one segment comprises identifying at least a section or a portion of one or more fragments of the anterior table fracture.

16. The surgical method of claim 14, wherein applying a pressure with the tip end of the surgical instrument to reduce the at least one segment comprises reducing one or more inferior or medial segments prior to reducing one or more superior or lateral segments.

17. The surgical method of claim 14, wherein forming the opening to the frontal sinus involves frontal sinusotomy.

18. The surgical method of claim 14, wherein the forming the opening to the frontal sinus involves an approach through a trephine or lynch incision.

19. The surgical method of claim 14, wherein an endoscope is used to visualize while introducing the surgical instrument.

20. The surgical method of claim 14, wherein the surgical instrument is selected from a kit of surgical instruments comprising individual ones of a plurality of surgical instruments, wherein each of the individual surgical instruments differ in at least one dimension from the others.
